(19) United States
(12) Patent Application Publication
(10) Pub. No.: US 2025/0064452 A1
(43) Pub. Date: Feb. 27, 2025

(54) COUPLEABLE WOUND CLOSURE MECHANISM AND METHODS OF MANUFACTURING AND USING SAME

(71) Applicant: SUTUREGARD Medical Inc., Corvallis, OR (US)

(72) Inventors: William Lear, Corvallis, OR (US); Daniel A Ladizinsky, Lake Oswego, OR (US)

(73) Assignee: SUTUREGARD Medical Inc., Corvallis, OR (US)

(21) Appl. No.: 18/947,087

(22) Filed: Nov. 14, 2024

Related U.S. Application Data
(63) Continuation of application No. PCT/US2023/020911, filed on May 4, 2023.
(60) Provisional application No. 63/370,385, filed on Aug. 4, 2022.
(60) Provisional application No. 63/342,068, filed on May 14, 2022.

(51) Int. Cl. A61B 17/08

(52) U.S. Cl. CPC A61B 17/085

(57) ABSTRACT

A wound closure device comprising a first portion including a first elongated support layer, a first non-woven layer coupled to the first elongated support layer, and a first fastening member coupled to the first elongated support layer and at least partially aligned with the first non-woven layer.

### CROSS-REFERENCE TO RELATED APPLICATIONS

This application claims priority from International patent application No. PCT/US23/20911, filed on May 4, 2023, which claims the benefit of the filing date of U.S. Provisional Patent Application No. 63/342,068, filed May 14, 2022, and U.S. Provisional Patent Application No. 63/370,385, filed Aug. 4, 2022, the disclosure of each is hereby incorporated by reference as if fully disclosed herein.

### FIELD OF THE DISCLOSURE

The present disclosure relates generally to wound closure and methods and devices for improving same. More specifically, the present disclosure relates to a coupleable wound closure mechanism and methods of using same.

### BACKGROUND OF THE DISCLOSURE

Sutures are stitches used to close open wounds and/or surgical incisions of a patient. A medical practitioner generally uses a needle with an attached thread to substantially sew two adjacent sections of skin together to close the wound or incision. Surgical knots are often used to secure the sutures and ensure proper healing. Sutures and surgical knots contacting the skin can be inflammatory and/or become “ingrown” and actually impede healing of the wound or incision. Additionally, complications may arise if the suture is tied too tightly or too loosely. Moreover, traditional techniques may leave unsightly “track marks.”

Closure may be difficult, especially in high-tension areas of the skin, such as where skin overlies the shoulder, knee, angle of the mandible, etc. Further, there may be excessive tension on closures where an excisional defect is present in the skin. When suture is placed under excessive tension to close such wounds, the suture itself can slice through the skin (“cheesewiring”).

Thus, there exists a need for suture devices that improve upon and advance the known suturing and closure mechanisms techniques.

### SUMMARY OF THE DISCLOSURE

In some embodiments, a wound covering includes a first portion including an elongated support layer a non-woven layer coupled to the elongated support layer a first fastening member coupled to the elongated support layer and at least partially overlapping with the non-woven layer, and a second portion including an elongated support layer having a first side and a second side, a non-woven layer coupled to the elongated support layer on the first side, a second fastening member coupled to the elongated support layer on the second side, the second fastening member being configured and arranged to couple to the first fastening member of the first portion.

### BRIEF DESCRIPTION OF THE DISCLOSURE

Various embodiments of the presently disclosed devices and wound closure mechanisms are disclosed herein with reference to the drawings, wherein:

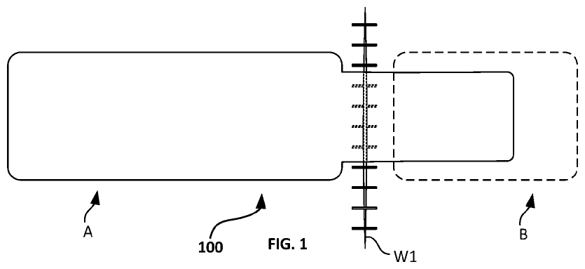

FIG. 1 is schematic top view of a coupleable wound closure mechanisms having two portions according to one embodiment of the present disclosure;

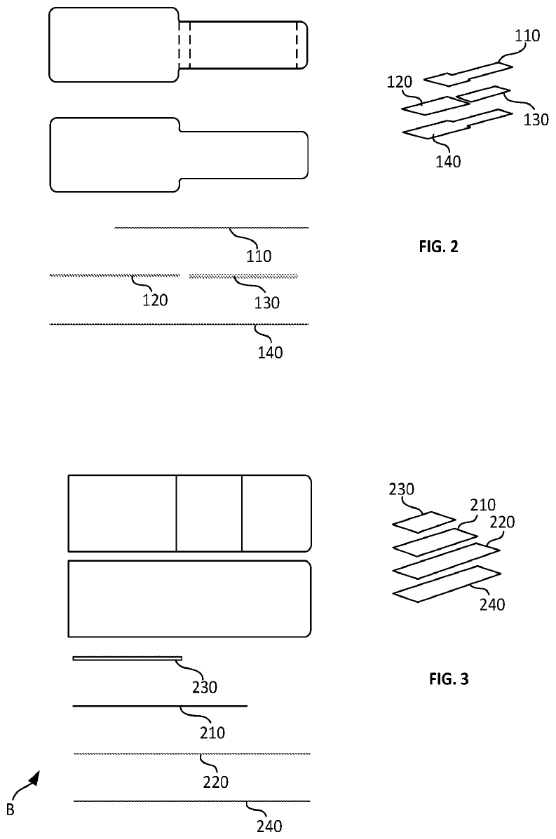

FIG. 2 are schematic top and exploded perspective views of a first portion of a coupleable wound closure mechanism according to one embodiment of the present disclosure;

FIG. 3 are schematic top and exploded perspective views of a second portion of a coupleable wound closure mechanism according to one embodiment of the present disclosure;

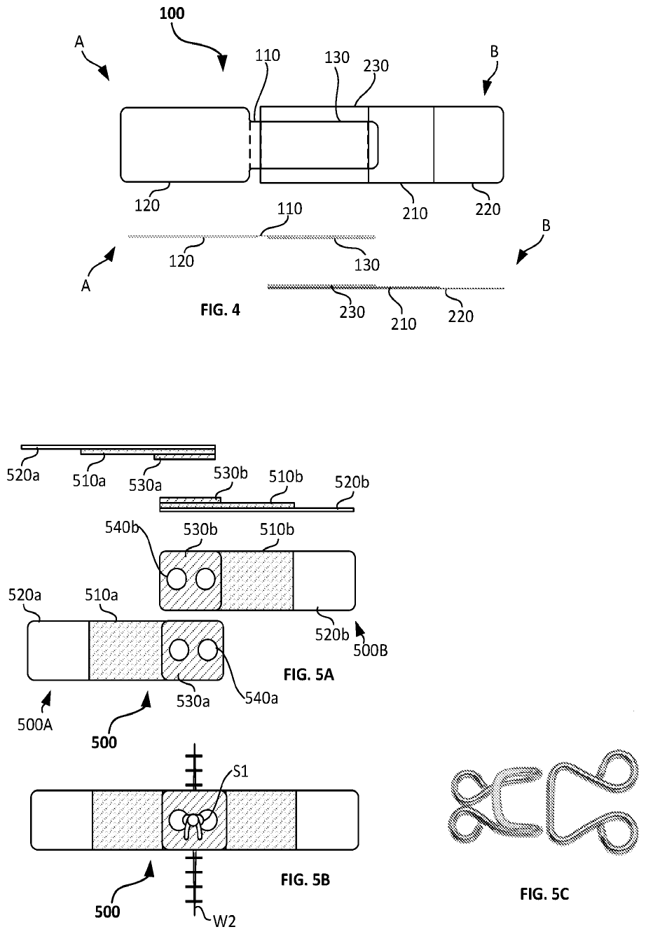

FIG. 4 are schematic top and side views of a coupleable wound closure mechanism according to one embodiment of the present disclosure;

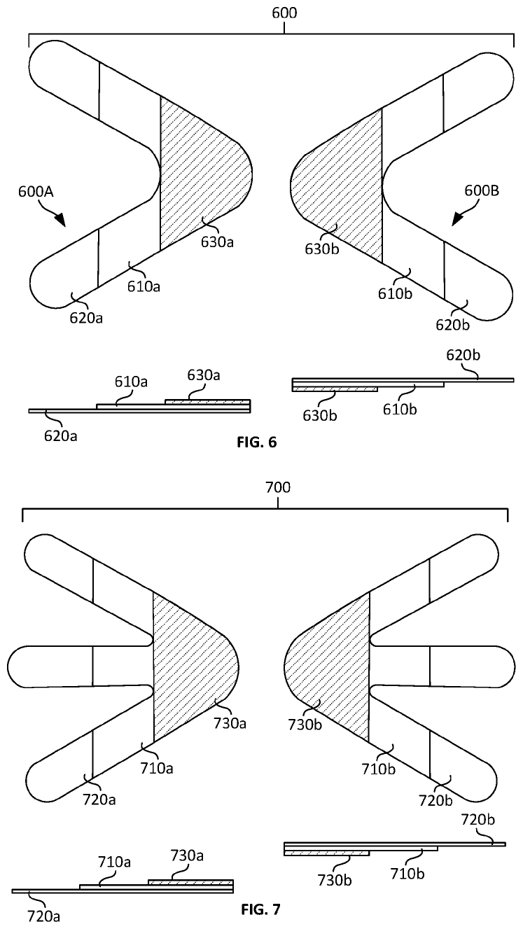

FIGS. 5A-B illustrate another coupleable wound closure mechanism disposed as a linear strip according to another embodiment;

FIG. 5C illustrates an alternative fastening member system;

FIGS. 6-7 illustrate wound closure mechanisms with V-shaped and Trident-shaped configurations;

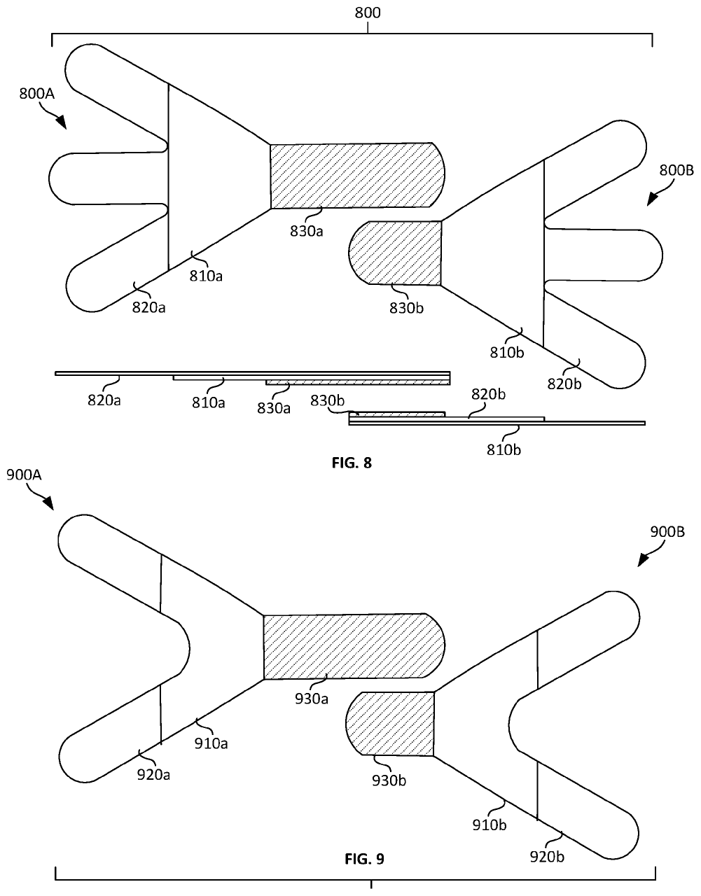

FIGS. 8-9 illustrate wound closure mechanisms with lateral extensions;

FIGS. 10-12 illustrate wound closure mechanisms with frangible multi-segment strips;

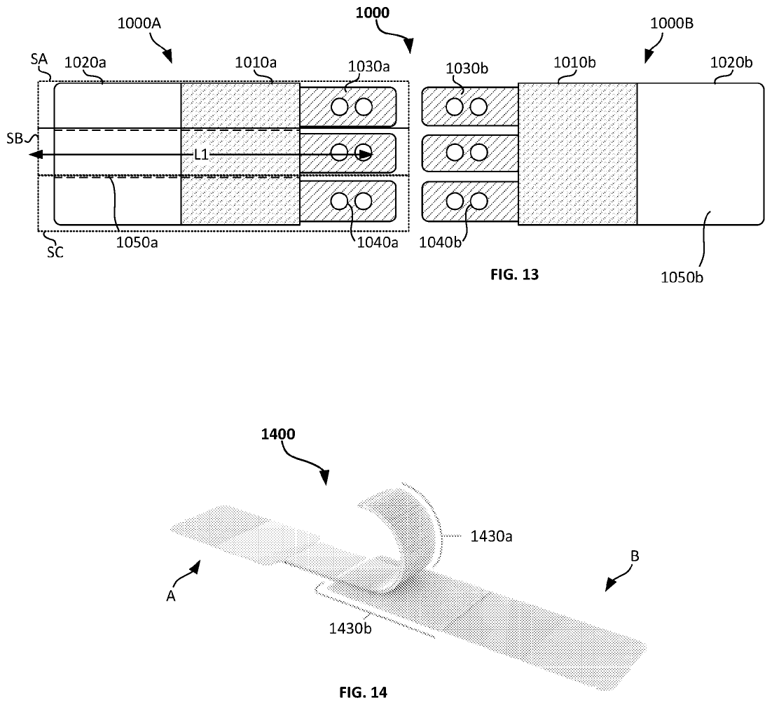

FIG. 13 illustrates some minor variations to the closure mechanism of FIGS. 10-12;

FIG. 14 illustrates another example of the wound closure device;

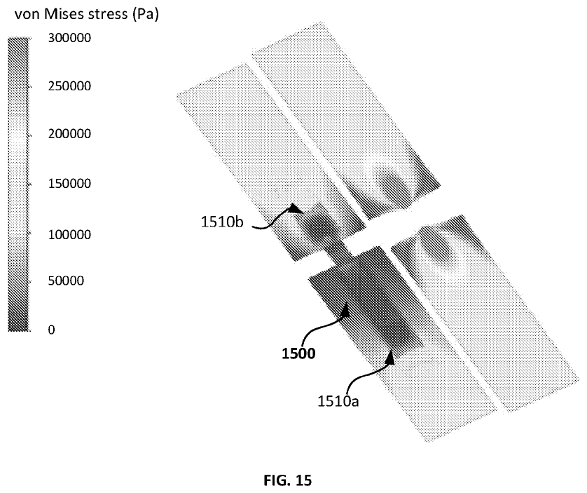

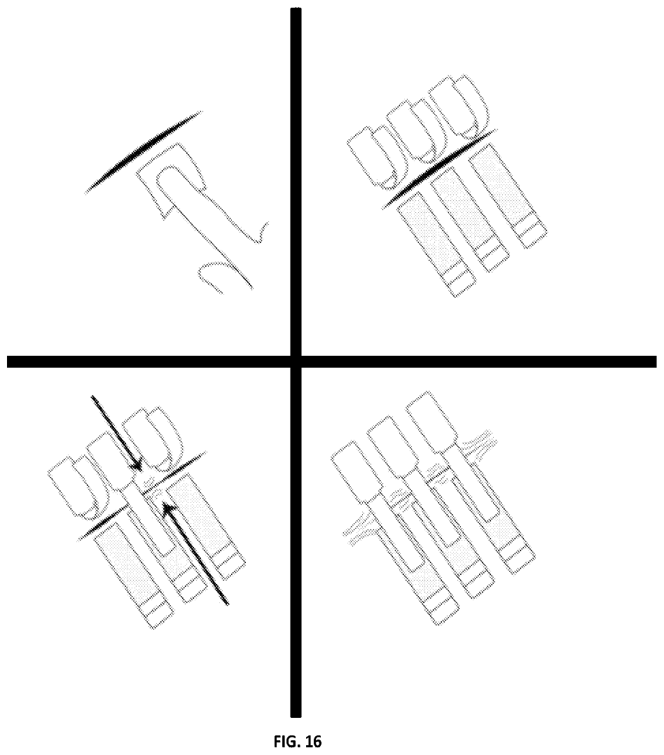

FIG. 15 illustrate a finite-eliminate analysis illustrating the offloading of stress from a closed wound disposed below one example of the wound closure device;

FIG. 16 illustrates the steps of a method of using a wound closure device; and

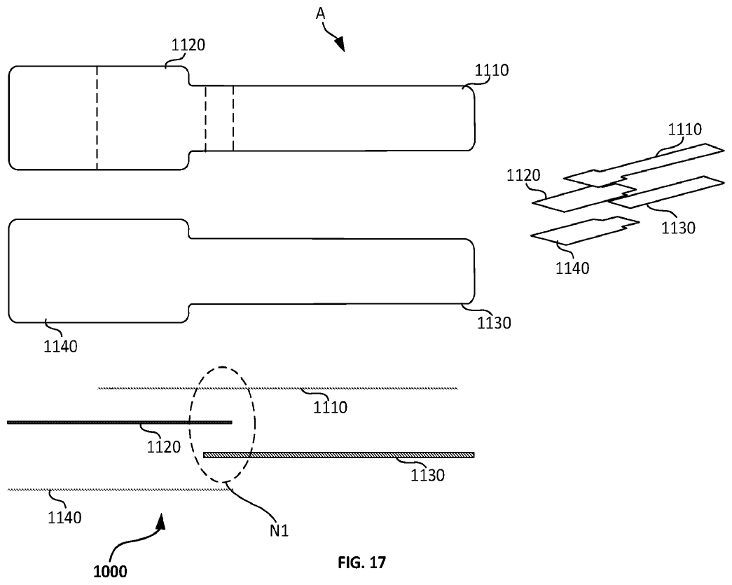

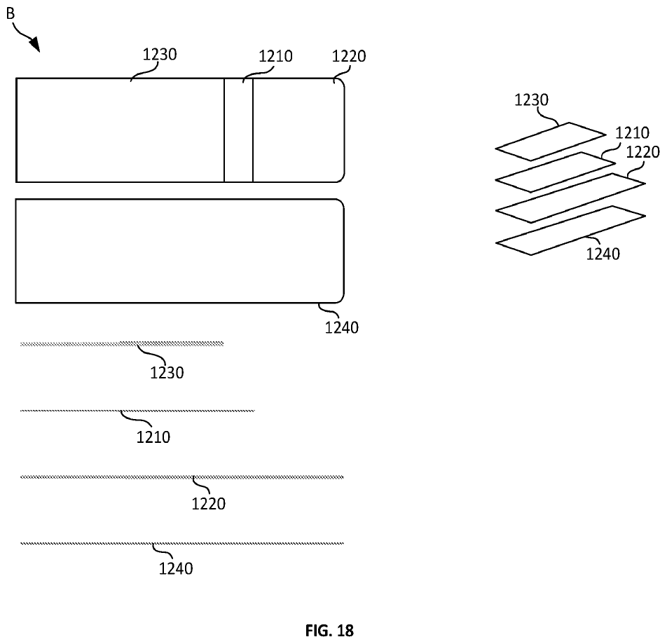

FIGS. 17-19 illustrate another example of a wound closure device.

Various embodiments of the present invention will now be described with reference to the appended drawings. It is to be appreciated that these drawings depict only some embodiments of the invention and are therefore not to be considered limiting of its scope.

### DETAILED DESCRIPTION

Despite the various improvements that have been made to wound closure devices, conventional methods suffer from some shortcomings as discussed above.

There therefore is a need for further improvements to the devices and methods used to help facilitate proper and quicker healing of a wound. Among other advantages, the present disclosure may address one or more of these needs.

FIG. 1 is a schematic top view of a two-part coupleable wound closure mechanism 100 that includes a first portion “A” and a second portion “B”. The two portions A,B, may be relatively similar in size (e.g., each may form half the device) or one may be larger than the other. As shown, portion A is substantially larger than portion B and is configured and arranged to extend over the wound edge “W1” and couple with at least part of portion B. As described in greater detail, each of the two portions may be laminated with layers of different materials to provide superior closure and comfort to the patient.

FIG. 2 are schematic top and exploded perspective views of a first portion “A” of a coupleable wound closure mechanism 100. In this example, first portion “A” will be described as the upper portion. Generally, first portion “A” includes a support layer 110, a non-woven layer 120, a first fastening member 130 and a removeable liner 140.

Support layer 110 may be formed of rigid or semi-rigid material such as a thermoplastic material such as polypropylene, polyethylene terephthalate, polyethylene (LDPE and HDPE), polymethylmethacrylate, polyethylene terepthalate glycol (PTG) such as 10 MIL or 20 MIL PETG or as low as 1 MIL PETG, polydimethyl siloxane, polyoxymethylene, polycarbonate, polyamide and nylon, polyvinyl chloride, polyphenylene sulfide, acrylonitrilebutadienestyrene, polystyrene, polytetrafluoroethylene or polyurethane. Preferably the thermoplastic material may have a suitable melting temperature.

In some examples, non-woven layer 120 includes a suitable non-woven material that prevents the absorption of blood and/or fluids, such as a polyurethane material. In some examples, non-woven layer 120 may have an adhesive lower surface that will be in contact with the skin. Alternatively, both surfaces of non-woven layer 120 may have an adhesive. The material of non-woven layer 120 may be isotropic (i.e., it has equal elasticity in any direction along its plane). Alternatively, the material of non-woven layer 120 may be anisotropic (i.e., it has variable elasticity in at least two directions along its plane). For example, non-woven layer 120 may have a first elasticity along its longitudinal axis, and a second elasticity perpendicular to its longitudinal axis, the first elasticity being greater than the second elasticity, or vice versa.

First fastening member 130 may comprise, for example, one half of a 3M™ brand Medical Hook Fastener 7336 with a low profile and an adhesive on the surface facing support layer 110. The fastening member may include either the hook portion of the fastener or the loop portion of the fastener. As shown in FIG. 2, in at least some examples, first fastening member 130 and non-woven layer 120 are disposed side-by-side and separated from each other by a narrow gap. A removeable, printed bottom liner 140 may cover at least a part of, or all, of first fastening member 130 and non-woven layer 120 until use. In at least some examples, liner 140 may include printed C1S 54 lb paper or a glassine liner.

FIG. 3 are schematic top and exploded perspective views of a second portion “B” of a coupleable wound closure mechanism 100. In this example, second portion “B” will be described as the lower portion. Generally, second portion “B” includes a support layer 210, a non-woven layer 220, a second fastening member 230 and a removeable liner 240.

Support layer 210 may be formed the same or different material than support layer 110 of portion “A”. In this example, support layer 210 is a generally rectangular piece of polyethylene.

In some examples, support layer 210 may be disposed on, and coupled to, a larger rectangular non-woven layer 220, which includes a suitable non-woven material that prevents the absorption of blood and/or fluids, such as a polyurethane material. In some examples, non-woven layer 220 may have an adhesive lower surface that will be in contact with the skin. Alternatively, both surfaces of non-woven layer 220 may have an adhesive. The material of non-woven layer 220 may be isotropic or anisotropic as previously discussed above with reference to non-woven layer 120.

Second fastening member 230 may comprise, for example, a complementary half of a 3M™ brand Medical Hook Fastener 7336 with a low profile and an adhesive on the surface facing support layer 210 (e.g., either the hook portion of the fastener or the loop portion of the fastener that complements that of first fastening member 130). Here, a removeable, printed bottom liner 240 may cover substantially at least a part or all of non-woven layer 220 until use. In at least some examples, liner 240 may include printed C1S 54 lb paper or a glassine liner.

In use, the physician may remove liner 240 from portion “B” and place the adhesive surface of portion B down on the patient's skin on one side of the wound, for example, with the second fastening member 230 being disposed closer to the wound margin and facing upward. Portion “B” may be spaced from the wound margin by 0.5 and 3 cm. The physician may then remove liner 140 from portion “A” and adhere portion “A” to the patient's skin on the opposing end of the wound, while extending the side of portion “A” that has the first fastening member 130 over the wound until it approximately overlaps with second fastening member 230. The first and second fastening members 130,230 may be coupled together.

FIGS. 5A-B are schematic top and side views of a linear strip closure device 500. In this example, closure device 500 includes a first portion 500A and a second portion 500B. First portion 500A may include an elongated support layer 510a, a non-woven layer 520a and a first fastening member 530a. Second portion 500B may include a second elongated support layer 510b, a non-woven layer 520b, and a second fastening member 530b, configured and arranged to couple with the first fastening member 530a. Optionally, one or more eyelets 540a,540b may be defined in either or both portions 500A,500B for receiving a suture so that the portions couple together via the fastening members as well as a suture S1 over wound W2. It will be understood that hook-and-loop configurations may be used for the first and second fastening members as previously described.

It will be understood that hook-and-loop configurations are disclosed herein, with the hook and the loop members being interchangeable between the first and second portions (e.g., the hook members may face down from the first portion or may face up from the second portion, and the loop member may take the opposite arrangement). In addition to hook-and-loop members, hook-and-eye closures (FIG. 5C) or two opposing hook-and-hook members may be integrated into the first and second portions to fasten the two elements together. Adhesives and other coupling members are also contemplated in addition to, or instead of, the hook and loop members.

FIG. 6 are schematic top and side views of a V-shaped closure device 600. In this example, closure device 600 includes a first portion 600A and a second portion 600B. First portion 600A may include an elongated support layer 610a, a non-woven layer 620a and a first fastening member 630a. Second portion 600B may include a second elongated support layer 610b, a non-woven layer 620b, and a second fastening member 630b, configured and arranged to couple with the first fastening member 630a. It will be understood that hook-and-loop configurations may be used for the first and second fastening members as previously described. An optional removable liner (not shown) may also be disposed on skin-contacting surfaces. A similar arrangement is shown in FIG. 7, except that a trident configuration is shown with three rays instead of two. In this example, similar elongated support layers 710a,710b, non-woven layers 720a,720b and first and second fastening members 730a,730b are shown as well as the layering of each of three laminated segments.

In FIGS. 8-9, mechanisms 800,900 are similar to those of FIGS. 6-7 except that the first and second fastening members 830a,830b and 930a,930b are disposed on lateral protruding extensions. As shown in FIG. 8, elongated support layers 810a,810b, non-woven layers 820a,820b and first and second fastening members 830a,830b are shown with protruding lateral extension and a trident configuration is illustrated. FIG. 9 is substantially similar to the configuration of FIG. 8, except that a V-shaped configuration is shown with two rays instead of three.

FIGS. 10-12 are schematic top views of frangible multi-segment strip closure device 1000. In this example, closure device 1000 includes a first portion 1000A and a second portion 1000B, the first and second portions being similar in the configurations, and layering of materials (including the order) described above. Specifically, first portion 1000A may include an elongated support layer 1010a, a non-woven base layer 1020a and multi-segment fastening member 1030a. In the example shown, three separate fastening members 1030 are formed, the three segments being spaced from one another. In this example, first portion 1000A may be tearable or frangible so that it can be sectioned or divided into one or more strips by cutting the portion along the axis L1. In some examples, first portion 1000A may include one or more weakened regions 1050a (marked with a dashed line) to allow the user to divide the first portion 1000A into an appropriate (e.g., smaller) shape. In this example, the first portion 1000A of FIG. 10 is initially provided in three segments, SA, SB, SC. In at least some examples, the three segments are divided by weakened regions 1050a, which may include one or more scored lines, one or more notches, or perforations. For example, the user may decide, based on the size, shape and/or location of the wound, that two strips or segments would be beneficial. In that case, the user may cut the device along one of the perforations to keep two segments and discard the third segment, effectively reducing the width of the first portion. Alternatively, the user may decide to use a single segment. In some examples, the user may use scissors to separate the device along the weakened regions 1050a. Alternatively, the weakened regions 1050a may be tearable by simply pulling the segments apart. Though a first portion 1000A is shown having three segments, it will be understood that the first portion 1000A may be manufactured with four, five, six, seven, eight, nine, ten, eleven, or a dozen or more segments defined by perforations therebetween.

Second portion 1000B may include a second elongated support layer 1010b, a non-woven layer 1020b, and a unitary second fastening member 1030b, configured and arranged to couple with the first fastening member 1030a. The materials and order of layers of section portion 1000B may be similar to those described above with reference to FIGS. 2-4 and/or 5-6. Second portion 1000B may be separable into different segments and may include second weakened regions 1050b similar to weakened regions 1050a.

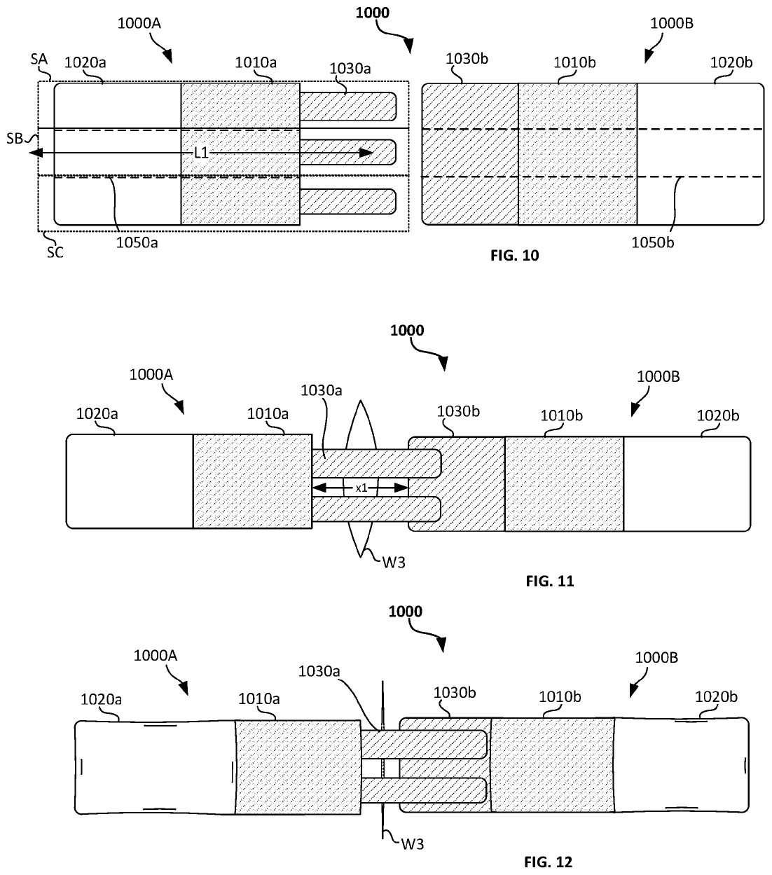

As shown in FIG. 11, the user has elected to use only two segments and has discarded a third segment from the device. It will be understood that, where “fastening members” are described, hook-and-loop configurations such as those disclosed herein may be used, with the hook and the loop members being interchangeable between the first and second portions (e.g., the hook members may face down from the first portion or may face up from the second portion, and the loop member may take the opposite arrangement). Adhesives and other coupling members are also contemplated in addition to, or instead of, the hook and loop members. Thus, the first and second portions may provide the user with increased optionality by being easily tailored to the required size.

In FIG. 11, the first and second portions 1000A, 1000B are aligned and disposed a select first distance “x1” apart on opposite sides of wound W3, but they are not yet coupled together (e.g., the hook-and-loop portions are mated together). With the two portions in place, the operator may stretch the portions slightly to bring fastening members 1030a over a greater portion of second fastening member 1030b. In doing so, portions of the two portions, 1000A, 1000B will stretch, but to varying degrees. In one example, non-woven base layer 1020a will have the lowest stiffness in first portion 1000A and stretch the most under the tension of closure as shown in FIG. 12. This feature of the stretchability of the trailing edge may prevent or lower the possibility of edge trailing edge blistering. In some examples, elongated support layer 1010a may stretch less than non-woven base layer 1020a, and fastening members 1030a may exhibit the lowest, or no stretching at all.

Similarly, non-woven base layer 1020b may have the lowest stiffness in first portion 1000B and stretch the most under the tension of closure to prevent or lower the possibility of edge trailing edge blistering. Elongated support layer 1010b may stretch less than non-woven base layer 1020b, and fastening members 1030b may exhibit the lowest, or no stretching at all.

Optionally, instead of being unitary, fastening members 1030b may be formed in a plurality of individual members or fingers, equal in number to fastening members 1030a as shown in FIG. 13. In some examples, fastening members 1030a,1030b may also include one or more eyelets 1040a,1040b defined in either or both portions 1000A, 1000B for receiving a suture so that the portions couple together via the fastening members as well as a suture. In such embodiments, the eyelets 1040a,1040b may be spaced apart by approximately 12 mm measured from center to center to accommodate a common needle.

FIG. 14 illustrates one example of a wound closure mechanism 1400 including first portion “A” and second portion “B”, which are being coupled together. First portion “A” may include a support layer, a non-woven layer, a first fastening member 1430a (e.g., hooks) and a removeable liner as previously described. Second portion “B” may include a support layer, a non-woven layer, a second fastening member 1430b and a removeable liner. Some of these layers may be optional.

FIG. 15 illustrate a finite-element analysis showing von Mises stress values when a wound closure mechanism 1500 is subjected to a load. In this analysis, the wound closure mechanism 1500 is shown to offload up to 20N of stress from a closed wound and involves no punctures. Additionally, as shown, a reduced stiffness at the two trailing edges 1510a,1510b may help to decrease the risk of shearing and/or blistering. Without being bound by any particular theory, it is believed that classic wound healing occurs in three main stages: inflammatory, proliferative and remodeling. The proliferative stage typically begins after the inflammatory stage and it is in the proliferative stage where collagen is laid down and breaking strength is accrued. It is believed that stress offload reduces areas of micro (or macro) ischemia. Ischemic damage, in turn, may prolong the inflammatory stage so that it is possible that use of the disclosed devices may shorten the inflammatory stage and allow wounds to heal faster than they would otherwise. In addition, use of the disclosed devices may minimize scarring and reduce wound complications including dehiscence.

FIG. 16 illustrates the general process of using wound closure mechanisms of the present disclosure. Specifically, the area around the wound can first be prepared and the surgeon or user may close the wound with sutures, staples and/or glue as per routine. Once prepared, the two portions of the wound closure mechanism may be adhered on opposing (e.g., contralateral) sides of a wound to clean dry skin. Slight tension may be applied to the strap and the two portions can be coupled together by bringing the fastening members together (e.g., by placing the hook portion down on, and in contact with the loop portion). The wound closure mechanism may be kept in place to support closure for up to three weeks. Additionally, the wound closure mechanism may be easily adjusted per indication or surgeon preference throughout the healing period. In some examples, the wound closure mechanism may be used without dermal sutures. In some examples, the wound closure mechanism is the initial step in the wound closure process or can be solely used to complete the entire process of opposing wound edges.

One variation of this disclosed wound closure mechanism is shown in FIGS. 17-19. In FIG. 17, schematic top and exploded perspective views of a first portion “A” of a coupleable wound closure mechanism 1000 are shown. In this example, first portion “A” will be described as the upper portion. Generally, first portion “A” includes a support layer 1110, a non-woven layer 1120, a first fastening member 1130 and a removeable liner 1140.

In this example of portion “A”, support layer 1110 may comprise 1 MIL PET with 3M™ Medical Transfer Adhesive 1524, non-woven layer 1120 may include a suitable non-woven material (e.g., DermaMed DM-2001, 3M™ Medical Tape 1776, MH 93630 or the like), first fastening member 1130 may include 3M™ Medical Hook Fastener 7336 or equivalent, and removeable liner 1140 may include printed C1S 54 lb paper or glassine liner. Other materials are possible such as those previously described. Moreover, the arrangement of the layers is shown as well as those particular axial overlaps between the various layers. Specifically, note the overlap between combinations of support layer 1110, non-woven layer 1120, and first fastening member 1130, especially at the reduced neck area “N1”.

In this example of portion “B”, the component includes a support layer 1210, a non-woven layer 1220, a second fastening member 1230 and a removeable liner 1240. The materials for portion “B” may include any of the examples described above. In some embodiments of portion “B”, support layer 1210 may comprise 1 MIL PET with 3M™ Medical Transfer Adhesive 1524, non-woven layer 1220 may include a suitable non-woven material (e.g., DermaMed DM-2001, 3M™ Medical Tape 1776, MH 93630 or the like), second fastening member 1230 may include 3M™ Medical Loop Fastener 7337 or equivalent, and removeable liner 1240 may include printed CIS 541b paper or glassine liner.

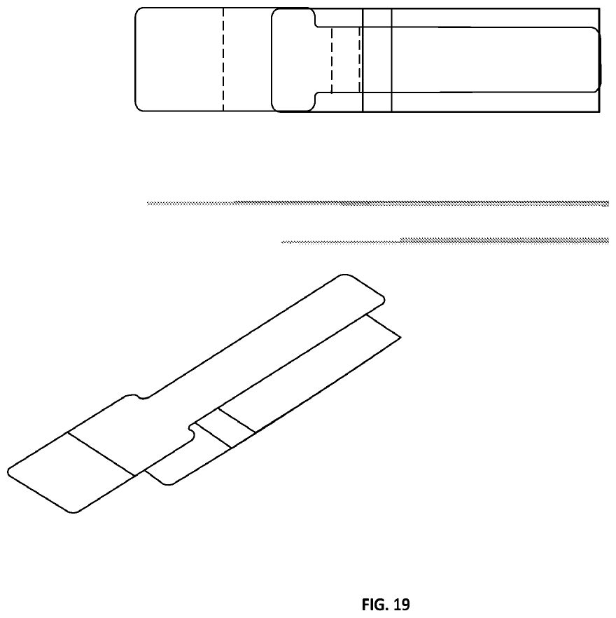

FIG. 19 illustrates the overlaying of the two portions on top of one another in use, the orientation of the two portions (and specifically the orientation of the hooks and loops), and the relative size of the two portions relative to one another.

The closures devices, systems, and methods described herein may be used to reduce or eliminate the use of sutures. Additionally, the devices disclosed herein may be capable of allowing a physician to apply a large force without damaging nearby tissue, and in some cases may be used to avoid the usage of skin grafts to close relatively large wounds. Alternatively, sutures, staples or other closure mechanisms may be used in conjunction with these devices, and may be implemented before or after the application of the coupleable wound closure mechanism for increased closure strength.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with others of the described embodiments.

1. A wound closure device comprising:
a first portion including:
a first elongated support layer;
a first non-woven layer coupled to the first elongated support layer; and
a first fastening member coupled to the first elongated support layer and disposed adjacent the first non-woven layer.

2. The wound closure device of claim 1, further comprising:
a second portion including:
a second elongated support layer having a first side and a second side;
a second non-woven layer coupled to the second elongated support layer on the first side; and
a second fastening member coupled to the second elongated support layer on the second side.

3. The wound closure device of claim 2, wherein the second fastening member of the second portion is configured and arranged to couple to the first fastening member of the first portion.

4. The wound closure device of claim 3, wherein the first fastening member comprises hooks, and the second fastening member comprises loops, the hooks being releasably coupleable to the loops.

5. The wound closure device of claim 3, wherein the second fastening member comprises hooks, and the first fastening member comprises loops, the hooks being releasably coupleable to the loops.

6. The wound closure device of claim 2, wherein the first and second elongated support layers comprise a thermoplastic material.

7. The wound closure device of claim 2, wherein the first and second elongated support layers comprise a rigid material.

8. The wound closure device of claim 1, further comprising an adhesive layer disposed on the first non-woven layer.

9. The wound closure device of claim 8, further comprising a first removeable liner at least partially covering the first non-woven layer.

10. A wound closure device comprising:
a first portion including:
a first elongated support layer;
a first non-woven layer coupled to the first elongated support layer;
at least one first fastening member coupled to the first elongated support layer; and
at least one first weakened region disposed along an axis of the first portion so that the first portion may be divided into at least two segments.

11. The wound closure device of claim 10, further comprising:
a second portion including:
a second elongated support layer having a first side and a second side;
a second non-woven layer coupled to the second elongated support layer on the first side;
at least one second fastening member coupled to the second elongated support layer on the second side; and
at least one second weakened region disposed along an axis of the second portion so that the second portion may be divided into at least two segments.

12. The wound closure device of claim 11, wherein the second fastening member is configured and arranged to couple to the first fastening member of the first portion.

13. The wound closure device of claim 11, wherein the at least one first fastening member comprises hooks, and the at least one second fastening member comprises loops, the hooks being releasably coupleable to the loops.

14. The wound closure device of claim 11, wherein the at least one second fastening member comprises hooks, and the at least one first fastening member comprises loops, the hooks being releasably coupleable to the loops.

15. The wound closure device of claim 11, wherein the first and second elongated support layers comprise a thermoplastic material.

16. The wound closure device of claim 11, wherein the first weakened region includes a first scored line extending across the first elongated support layer, the first non-woven layer and the at least one first fastening member, and wherein the second weakened region includes a second scored line extending across the second elongated support layer, the second non-woven layer and the at least one second fastening member.

17. The wound closure device of claim 10, wherein the at least one first fastening member includes a plurality of fastening members spaced from one another.

18. The wound closure device of claim 11, wherein the at least one second fastening member includes a plurality of fastening members spaced from one another.

19. A method of closing a wound comprising:
adhering two portions of a wound closure device on opposing sides of the wound, (i) a first portion of the two portions including a first elongated support layer, a first non-woven layer coupled to the first elongated support layer, and a first fastening member coupled to the first elongated support layer and at least partially axially overlapping with the first non-woven layer, and (ii) a second portion of the two portions including a second elongated support layer having a first side and a second side, a second non-woven layer coupled to the second elongated support layer on the first side, and a second fastening member coupled to the second elongated support layer on the second side; and
coupling the first fastening member to the second fastening member.

20. The method of claim 19, further comprising the steps of:
closing the wound with sutures, staples and/or glue prior at least one of (i) adhering two portions of the wound closure device or (ii) coupling the first fastening member to the second fastening member; and
offloading stress from the closed wound with the wound closure device.
